(19) United States
(12) Patent Application Publication
(10) Pub. No.: US 2007/0213298 A1
(43) Pub. Date: Sep. 13, 2007

(54) Omega 3

(75) Inventors: Pal Rongved, Oslo (NO); Jo Klaveness, Oslo (NO)

Correspondence Address: SUTHERLAND ASBILL & BRENNAN LLP, 999 PEACHTREE STREET, N.E., ATLANTA, GA, 30309, US

(73) Assignee: UNIVERSITETET I OSLO, Oslo (NO)

(21) Appl. No.: 11/672,249

(22) Filed: Feb. 7, 2007

(30) Foreign Application Priority Data
Feb. 7, 2006 (GB) 0602450
Sep. 14, 2006 (GB) 0618128

(51) Int. Cl. A61K 31/70

(52) U.S. Cl. 514/62

(57) ABSTRACT

A process for the preparation of a water soluble unsaturated fatty acid salt from a crude composition comprising at least one non-water soluble or sparingly water soluble unsaturated fatty acid or salt thereof, said process comprising: adding to said crude composition in the presence of water at least one amino alcohol compound so as to form a water soluble amino alcohol salt of said acid or salt; separating an aqueous phase; and optionally isolating said salt from said aqueous phase.

### TECHNICAL FIELD

This invention relates to a process for preparing water soluble unsaturated fatty acid salts, e.g. water soluble omega-3 fatty acid salts and/or water soluble omega-6 fatty acid salts from a crude composition comprising the unsaturated fatty acid, in particular a natural source of the fatty acid, e.g. a plant or animal oil such as a marine oil. The invention also relates to certain new water soluble salts and mixtures thereof which can be prepared by the process of the invention. In particular, the invention concerns a process in which water soluble monoamino alcohol or polyamino alcohol salts of omega-3 fatty acids are formed and in which these compounds are used in health promoting supplements or as drug formulations.

### BACKGROUND OF THE INVENTION

Omega-3 and omega-6 fatty acids are fatty acids essential to human health but ones which cannot be manufactured by the body. For this reason, omega-3 fatty acids must be obtained from food sources and can be found in fish and certain plant oils. It is important to maintain an appropriate balance of omega-3 and omega-6 (another essential fatty acid) in the diet as these two substances work together to promote health. Omega-3 and omega-6 fatty acids play a crucial role in brain function as well as normal growth and development for example.

There are three major types of unsaturated fatty acids that are ingested in foods and used by the body: the omega-6 fatty acid alpha-linolenic acid (ALA), and the omega-3 fatty acids eicosapentaenoic acid (EPA) and docosahexaenoic acid (DHA). Once eaten, the body converts ALA to EPA and DHA, the two types of omega-3 fatty acids more readily used by the body. Extensive research indicates that omega-3 fatty acids reduce inflammation and help prevent certain chronic diseases such as heart disease and arthritis. These essential fatty acids are highly concentrated in the brain and appear to be particularly important for cognitive and behavioural function. In fact, infants who do not get enough omega-3 fatty acids from their mothers during pregnancy are at risk for developing vision and nerve problems.

As mentioned previously, it is very important to maintain a balance between omega-3 and omega-6 fatty acids in the diet. For example, omega-3 fatty acids help reduce inflammation whereas most omega-6 fatty acids tend to promote inflammation. An inappropriate balance of these essential fatty acids contributes to the development of disease while a proper balance helps maintain and even improve health. A healthy diet should consist of roughly one to four times more omega-6 fatty acids than omega-3 fatty acids.

With the development of convenience foods and a general decline in the consumption of healthy foodstuffs such as fresh fish, fruit and vegetables, the typical American diet tends to contain 11 to 30 times more omega-6 fatty acids than omega-3 fatty acids and many researchers believe this imbalance is a significant factor in the rising rate of inflammatory disorders in the United States.

In contrast, however, the Mediterranean diet consists of a healthier balance between omega-3 and omega-6 fatty acids and many studies have shown that people who follow this diet are less likely to develop heart disease. The Mediterranean diet does not include much meat (which is high in omega-6 fatty acids) and emphasizes foods rich in omega-3 fatty acids including whole grains, fresh fruits and vegetables, fish, olive oil, garlic, as well as moderate wine consumption.

Thus, since their discovery in the 1970s, and the finding that the ratio of omega-3 to omega-6 acids is imbalanced in the diets of many individuals, omega-3 fatty acids or their derivatives have been made available to consumers as dietary supplements to try to restore the desired omega-3 to omega-6 balance. Omega-3 fatty acids or derivatives thereof are thus now taken routinely by many hundreds of thousands of individuals to prevent a variety of illnesses such as arthritis, cardiac infarction and stroke.

Omega-3 fatty acids are often provided to consumers in their naturally occurring triglyceride form. The Omega-3 fatty acid triglyceride or the free fatty acid itself are generally sourced from natural oils such as marine oils. Since it is difficult to isolate the omega-3 acids in high purity from marine oils, omega-3 supplements often possess an unpleasant fishy after taste which the consumer dislikes. It is also a major problem for many individuals, such as the elderly and children, to swallow the gelatine capsules used today to contain the omega-3 material. It would be useful therefore if omega-3 compounds could be isolated in sufficient purity that the after taste of fish can be removed. It would also be useful if the concentration of omega-3 compounds in any composition could be enriched relative to the natural source.

A particular problem with omega-3 fatty acids and long chain unsaturated fatty acids in general is that they are water insoluble and typical salts thereof, such as sodium salts thereof, are also water insoluble. This insolubility severely limits their bioavailability and hinders purification thereof. It would be very useful therefore to be able to obtain a water soluble omega-3 compound from a crude mixture containing omega-3 material, e.g. in its triglyceride form or free acid form.

Moreover, due to pollution in our sea and fresh waters, many marine oils now contain high levels of poisonous compounds such as dioxins, heavy metals, polyhalogenated flame retardants and PCB's (polychlorinated biphenyls). It would be useful if omega-3 fatty acids could be isolated from crude marine oils in such a way that these poisons are also eliminated.

It is also important that any separation method used is cheap and applicable on an industrial scale. The market for omega-3 supplements is vast but the margins on the product are small and products retail cheaply. Expensive chromatography techniques cannot therefore be used to isolate omega-3 acids from crude marine oils as the cost of such purifications would make the omega-3 too expensive to sell. Molecular distillation is used today to purify ethyl esters of EPA and DHA, e.g. as in the process for the drug Omacor®, but this is not useful for purifying crude marine oils.

### SUMMARY OF THE INVENTION

The inventors have surprisingly found a new process for the manufacture of a composition enriched in pure water soluble unsaturated fatty acid salts, especially omega-3 salts. In some embodiments, the process allows removal of shorter chain fatty acids and low molecular weight amines which contribute to fish taste and noxious smell and which do not contribute to the health benefits of omega-3 whilst also allowing the elimination of poisons such as dioxins, heavy metals, flame retardants and PCB's leaving a composition highly enriched in omega-3 fatty acid salts, in particular polyamino alcohol salts. The process can even eliminate the presence of cholesterol. Moreover, the water soluble unsaturated fatty acid salts or mixtures thereof can be isolated in solid form, e.g. powder, form making them ideal for formulation in pharmaceutical or nutraceutical dosage forms.

The present inventors have found a way of converting a naturally occurring crude fatty acid derivative containing oil such as a marine oil into a composition containing water soluble omega-3 salts which can be used directly in the manufacture of medicaments for the treatment and prevention of disease. The process is cheap and simple to carry out and does not require expensive purification procedures.

In its broadest embodiment the process of the invention allows the formation of water soluble salts of unsaturated fatty acids and hence enables the purification of a crude mixture containing the desired acid by extraction of the water soluble salt into the aqueous phase leaving non water soluble impurities behind. The invention also teaches the formation of new water soluble salts, e.g. those based on polyamino alcohol compounds and mixtures of water soluble unsaturated fatty acid salts. Salts of omega-3 fatty acids are not new. For instance, WO96/33155 describes meglumine (N-methyl glucamine) salts of certain specific prepurified omega-3 fatty acids and suggests various therapeutic applications thereof. The present invention provides however, the first process for taking the unsaturated fatty acid in crude form, i.e. in the presence of impurities and preparing a purified water soluble salt thereof and also provides the first disclosure of a mixture of water soluble unsaturated fatty acid salts. In this regard, it is most surprising that salt mixtures can be isolated according to the present invention as crystalline or semi-crystalline materials. When mixtures of compounds are present, the recovery of crystalline material is unusual as the presence of more than one compound causes a melting point reduction and the formation of oily material.

Thus, viewed from one aspect the invention provides a process for the preparation of a water soluble unsaturated fatty acid salt, e.g. a mixture of water soluble unsaturated fatty acid salts, from a crude composition comprising a non-water soluble or sparingly water soluble unsaturated fatty acid or salt thereof, said process comprising:

adding to said crude composition in the presence of water at least one amino alcohol compound so as to form a water soluble amino alcohol salt of said acid or salt;

separating the aqueous phase; and

optionally isolating said salt from said aqueous phase.

Viewed from another aspect, the invention provides a process for the preparation of a mixture of water soluble unsaturated fatty acid salts from a non-water soluble or sparingly water soluble mixture of unsaturated fatty acids or salts thereof, said process comprising:

adding to said mixture of non-water soluble or sparingly water soluble unsaturated fatty acids or salts thereof in the presence of water at least one amino alcohol compound so as to form water soluble amino alcohol salts of said acids or salts.

separating the aqueous phase; and

optionally isolating said salt from said aqueous phase.

Viewed from another aspect, the invention provides a process for the preparation of a water soluble unsaturated fatty acid salt from a non-water soluble or sparingly water soluble unsaturated fatty acid or salt thereof said process comprising:

adding to said non-water soluble or sparingly water soluble unsaturated fatty acid or salt thereof in the presence of water at least one polyamino alcohol compound so as to form a water soluble polyamino alcohol salt of said acid or salt;

separating the aqueous phase; and

optionally isolating said salt from said aqueous phase.

The first process of the invention allows the formation of a water soluble unsaturated fatty acid salt or mixture of salts from a crude composition containing non water soluble unsaturated fatty acids or salts and thereby allows purification of the fatty acid by separating it from non-water soluble impurities. By purification is meant therefore that the target material, i.e. the water insoluble amino alcohol salt of the unsaturated fatty acid, is separated from at least one impurity present in the crude composition, preferably a plurality of impurities, especially a plurality of water soluble impurities such as cholesterol and the poisons mentioned below. The crude composition therefore contains a non water soluble or sparingly water soluble unsaturated fatty acid or salt as well as at least one impurity.

The processes of the invention also enable the formation of a mixture of water soluble amino alcohol fatty acid salts by reaction of a water insoluble unsaturated fatty acid mixture or salt mixture in pure form and allows the formation of a polyamino alcohol fatty acid salt by reaction of an insoluble unsaturated fatty acid or salt in pure form. In any of these processes the non water soluble or sparingly water soluble unsaturated fatty acid compound is preferably in its COOH, form prior to amino alcohol compound addition.

These and other features of the present application will become apparent to one of ordinary skill in the art upon review of the following detailed disclosure when taken in conjunction with the several drawings and the appended claims.

### DETAILED DESCRIPTION

The discussion which follows assumes the non-water soluble or sparingly water soluble unsaturated fatty acid is in the COOH form but equally relates to the salt form if this is present in the starting material. Any non-water soluble or sparingly water soluble unsaturated fatty acid, preferably an omega-3 fatty acid, present in the crude composition or used in the process in general is preferably derived from a natural source such as a plant oil or an animal oil. Oils which contain unsaturated fatty acids, typically present as esters of the fatty acids, are well known in the art. Suitable plant oils include rapeseed oil, corn oil, soya oil, sunflower oil, vegetable oil and olive oil. Preferably however, the natural source of the unsaturated fatty acid is an animal oil such as tallow oil.

Highly preferably however, the source of the unsaturated fatty acid is a marine oil, such as a fish oil or krill oil. Crude marine oil used in this invention can be derived from any marine source such as fish, especially seawater fish such as tuna, sardines, salmon, mackerel, herring, trout, halibut, cod, haddock, catfish, sole etc. The use of oily fish is preferred. Ideally however, the crude marine oil will derive from marine mammals such as seals, walrus or sea lions, preferably seals or from krill. Seal oil has been found to be especially rich in omega-3 fatty acid compounds, e.g. of the order of 20-25 wt % and therefore forms an ideal starting material to form the crude composition of the invention. Seal oils are available from a variety of commercial sources.

The unsaturated fatty acid contains one or more double bonds. Preferably it is polyunsaturated. The crude composition on which the invention is carried out can comprise one such non-water soluble or sparingly water soluble unsaturated fatty acid or a mixture of such unsaturated fatty acids. Preferably, it contains a mixture of unsaturated fatty acids and hence the product of the process is a mixture of fatty acid salts. It will be appreciated that the crude composition might also contain saturated fatty acids as these are also present in naturally occurring unsaturated fatty acid sources.

Preferably, the non-water soluble or sparingly water soluble unsaturated fatty acid is an omega-3 fatty acid in which the double bond most distant from the carboxylic acid functionality is located at the third bond counted from the end (omega) of the carbon chain. The fatty acid may also be an omega-6 fatty acid where the double bond most distant from the carboxylic acid functionality is located at the sixth bond counted from the end (omega) of the carbon chain. A crude composition used in the invention, e.g. a crude marine oil is likely to contain a variety of omega-3 and omega-6 fatty acids. The invention therefore covers a process in which a mixture of water soluble omega-3 unsaturated fatty acid salts is produced.

The total concentration of omega-3 fatty acids or derivatives thereof in a crude oil varies depending on the natural source in question but, for example, in sea fish, the amount of the omega-3 compounds is approximately 25 wt %.

As noted above, fatty acids are normally present as a derivative of the free acid in naturally occurring sources. By derivative of an unsaturated fatty acid, e.g. omega-3 or omega-6 fatty acid is meant a salt, amide or ester thereof, or any other compound where the COON group is functionalised in such a way that it will return to an COOH group upon treatment, e.g. upon hydrolysis. Typically however, the fatty acid compounds in the crude oil are in the form of esters, especially triglycerides, i.e. the fatty acid derivative is a triglyceride.

Unsaturated fatty acids which can form part of the crude composition may be those of formula (I):
C₃(CH₂)ₙ—(CH═CH—CH₂)ₘ—(CH₂)ₛ—COOH (1)
wherein n, m and s are integers. e.g. of 1 to 10. Subscript n is preferably 1. Subscript m is preferably 2 to 8. Subscript s is preferably 1 to 6. Ideally, the carbon chain is linear although it is within the scope of the invention for the backbone to carry alkyl side chains such as methyl or ethyl. (For this formula DHA n=1, m=6 and s=1, for EPA n=1, m=5 and s=6. In ALA, n=4, m=2 and s=6).

Omega-3 fatty acids of use in the treatment or prevention of disease which can be converted into the amino alcohol salts of the invention are preferably those which contain at least 18 carbon atoms in the carbon backbone. Lower chain fatty acids (those of 17 carbon atoms or less in the backbone) appear to show fewer useful therapeutic effects, but can be useful in applications like fish or animal feed. Thus, preferred unsaturated fatty acids are those of formula (I′)
CH₃CH₂CH═CH—R—COOH (I′)
wherein R is a C₁₃₊ alkylene group (e.g. C₁₃₋₂₅) optionally containing 1 or more double bonds, preferably non-conjugated. Ideally, the R group is linear although it is within the scope of the invention for the backbone to carry alkyl side chains such as methyl or ethyl. The total number of carbon atoms in the chain is preferably 16 to 22. Moreover, R is preferably 13, 15, 17, 19 etc. i.e. the number of carbon atoms in the chain is preferably even. Whilst it will be appreciated that the omega 3 enriched composition made by the process of the invention will, most likely, contain a variety of different omega-3 based compounds, highly preferred compounds of formula (I) are C18, C20 and C22 compounds, ALA, DHA and EPA are especially preferred. (i.e. where R contains 13, 15 or 17 carbon atoms).

In a highly preferred embodiment, the fatty acids comprise DHA and EPA mixtures, i.e. the salts formed at the end of the claimed process include a mixture of DHA and EPA salts. The ratio of such salts may be 30:70 to 70:30, preferably 40:60 to 60:40 EPA/DHA.

The crude composition may also contain omega-6 fatty acids. Preferred omega-6 fatty acids, suitable for making the desired water soluble salts of the invention are those of formula (II):
CH₃CH₂CH₂CH₂CH₂═CH—R″—COOH (II)
wherein R″ is a C₅₊ alkylene group (e.g. C₁₀₋₂₂) optionally containing I or more double bonds. Ideally, the R″ group is linear although it is within the scope of the invention for the backbone to carry alkyl side chains such as methyl or ethyl.

The number of carbon atoms in R″ is preferably 10, 12, 14, 16 etc, i.e. the number of carbon atoms in the chain is preferably even. In a preferred embodiment the omega-6 fatty acid is a linoleic acid or conjugated linoleic acid.

Whilst it will be appreciated that the composition made by the process of the invention will, most likely, contain a variety of different omega 3 and 6 based compounds, highly preferred compounds of formula (II) are C18, C20 and C22 compounds.

The weight ratio of omega-3 to omega-d at the end of the process of the invention may be of the order 1:1 to 1:3.

Preferably, the salts formed by the process of the invention will have at least 10 carbon atoms, e.g. at least 12 carbon atoms, such as at least 14 carbon atoms in the fatty acid portion of the molecule, i.e. a fatty acid must comprise at least 10 carbon atoms.

Ideally compounds of formula (I), (I′) or (II) will be multiply unsaturated. e.g. contain 2 to 10 double bonds, especially 4 to 7 double bonds. Preferably double bonds are not conjugated either to each other or to the carbonyl functionality.

At least one, e.g. 2 or 3, preferably all double bonds are preferably in the cis configuration.

Crude oils contain a variety of fatty acids or derivatives thereof (e.g. esters thereof, in particular triglycerides) having differing carbon chain lengths and differing levels of unsaturation. Of course not all these fatty acids will be omega-3 unsaturated fatty acids, some will be omega-6 unsaturated, some may be saturated oils. It will be appreciated therefore that the water soluble composition manufactured during the process of the invention does not need to consist of omega-3 salts. Preferably, however water soluble omega-3 salts are present. Preferably, the concentration of omega-3 compounds in the final composition, relative to the crude oil, is significantly higher. What is important therefore is that the amounts of omega-3 in the final composition are enriched by formation into a water soluble salt and concurrent aqueous extraction. Enrichment occurs by removal of undesired impurities leaving a relatively higher concentration of omega-3 material.

The crude composition can comprise a polar solvent, e.g. a non aqueous solvent such as ethanol or DMSO or more preferably a non polar solvent such as hexane or toluene. Preferably however, water is the only polar solvent present during salt formation.

The process of the present invention may be carried out by forming a water soluble salt amino alcohol salt of the unsaturated fatty acid or salt present in the crude composition. The amino alcohol salt may contain a single amino group (a monoamino compound) or a plurality of amino groups (a polyamino compound). The amino alcohol compound also contains a hydroxyl functional group (i.e. an alcohol), preferably a plurality of hydroxyl groups. The amino alcohol compound can therefore contain one or more amino groups and one or more hydroxyl groups. Highly preferred amino alcohol compounds contain a plurality of hydroxyl groups with one amino group or a plurality of hydroxyl groups with a plurality of amino groups. The term polyamino alcohol used herein denotes a compound comprising a plurality of amino groups and at least one (preferably a plurality) of hydroxyl groups.

Suitable amino alcohol compounds might have 1 to 20 amino groups, e.g. one amino group or 5 to 15 amino groups. Suitable amino alcohol compounds might have 1 to 100 hydroxyls groups, e.g. 3 to 50 hydroxyl groups, e.g. 5 to 15. The amino groups may be primary, secondary or tertiary however secondary or especially primary amino groups are preferred.

The amino alcohol compound required to allow formation of the water soluble salts of the invention is thus preferably a hydroxylated amine with the general formula
((HO(R′)_{0/1})ₙ)R₁NHR₂)ₘ (III)
where n is an integer from 3 to 6, R′ is methyl, R₁ is a C1-20 alkylene group, R₂ is H or an C₁₋₆ alkyl side chain, preferably methyl and m is an integer from 1 to 20.

In the case of n=3, R′ is present and is methyl, R₁ is methyl and R₂═H, the hydroxyamine may be tris-(hydroxymethyl)-methylamine (Tris). In the case of n=5, R′ is absent, R₁ is a straight six carbon chain and R₂ is Me and m=1, the hydroxyamine may be meglumine.

Polyamino alcohol salts of the fatty acids are formed using polyamino alcohol compounds. Polyamino compounds are those containing two or more amino groups available for forming an ammonium salt with the carboxyl group of the fatty acid. Ideally, the polyamino compound employed will be a polyamino sugar. Such polyaminosugars will preferably contain a plurality of saccharide units along with a plurality of hydroxyl groups to assist the solubility of the formed salt.

Suitable polyaminosugars may be derived from chitin, especially chitosan. A suitable polyamino sugar may have a general formula
(C₆H₁₄NO₅)ₚ (IV)
where p is an integer of 2 or more, e.g. 5 to 15.

Especially preferred polyamino sugars are those available from FMC or Novamatrix. Chitosan is often supplied in acetate form. i.e. the amine groups are protected. The acetate can be removed using known ion exchange techniques to release the free polyamino form of chitosan.

Where a monoamino alcohol compound is used this may be meglumine, Tris or glucosamine. Manipulation of the reaction medium e.g. by varying its temperature, pH or ionic strength may enhance solubility of salts.

Mixtures of polyamino alcohol and monoamino alcohol salts may also be used, e.g. two polyamino salts, two monoamino salts or a mixture of polyamino and monoamino salts.

The amino alcohol compound forms a salt with the unsaturated fatty acid(s) or salt(s) present in the crude composition and becomes water soluble. The water soluble unsaturated fatty acid salt formed preferably has a solubility of at least 10 g/L of water. Preferably, the solubility of the formed salts is at least 15 g/L of water. The inventors have surprisingly found that the solubility of the fatty acid salts made by the processes of the invention is actually remarkably high. Solubilities of the salts of the invention may be as high as 40 g/L, more preferably 100 g/L, especially 250 g/L. The inventors have even discovered materials with solubilities of greater than 500 g/L.

Moreover, it has been established that mixtures of the water soluble fatty acid salts of the invention can also possess similar solubility levels.

The unsaturated fatty acids or salts in the crude composition are sparingly water soluble or non water soluble. Non water soluble materials preferably have solubilities of less than 0.1 g/L of water. Sparingly water soluble materials preferably have solubilities of less than 1 g/L of water, especially less than 0.5 g/L.

Thus, the previously non water soluble unsaturated fatty acids/salts or only sparingly water soluble fatty acids/salts become water soluble upon amino alcohol salt formation.

The actual salt formation reaction itself requires only that the fatty acid or salt, preferably the acid, be brought into contact with the amino alcohol compound in the presence of water. It will be appreciated that whilst it is most convenient to introduce the amino alcohol compound and water simultaneously (as an aqueous solution), the skilled man could add these separately. If water is already present in the crude composition it may be necessary simply to add the amino alcohol compound.

If a monoamino alcohol compound is employed the amount used may be stoichiometric or it may be used in excess. As polyamino alcohol compounds contain multiple amino groups, there is of course the possibility of multiple binding to the polyamino groups. Thus, whilst an excess of polyamino alcohol compound may also be used, it is envisaged that the ratio of polyamino alcohol compound to fatty acid will be of the order of 1:1 to 1:30, e.g. 1:2 to 1:10, especially 1:3 to 1:6.

The salt formation step can be heated or pressurised if desired although this is unnecessary as the salts form readily. The salt formation step can also involve stirring, sonication or more vigorous mixing processes such as centrifugation. It is preferred however that the pH of the reaction is initially adjusted to ensure that the fatty acids are in their acid form (rather than salt form COO⁻) to ensure that a salt with the amino alcohol compound can form more easily.

Once the water soluble fatty acids salts have been formed and extracted into the aqueous phase, this can be isolated from the organic phase. The water soluble fatty acid salts in the aqueous phase can then be manipulated however the skilled man sees fit. They can be isolated as discussed further below or the free acid form can be obtained again upon acidification of the aqueous phase and separation of the oil which forms. The resulting free fatty acids are however in a more pure form than in the starting material. Thus, purified fatty acid salt could then be manipulated in any way e.g. to form an ester (e.g. a glyceride) or another salt (e.g. a meglumine salt), phospholipid, amide, carbamate or they could be attached to polymers.

Certain water soluble salts of unsaturated fatty acids are new and these form a still further aspect of the invention.

Viewed from another aspect, the invention provides a water soluble tris-(hydroxymethyl)-methylamine salt or a polyamino alcohol salt of an unsaturated fatty acid, preferably an omega-3 or omega-6 fatty acid.

Viewed from another aspect the invention provides a mixture of water soluble amino alcohol salts of unsaturated fatty acids, preferably omega-3 or omega-6 fatty acids.

Viewed from another aspect, the invention provides a composition, e.g. a pharmaceutical composition, comprising a water soluble tris-(hydroxymethyl)methylamine salt or a polyamino alcohol salt of a unsaturated fatty acids, preferably an omega-3 or omega-6 fatty acid or a mixture of salts as hereinbefore described.

Viewed from another aspect the invention provides a water soluble tris-(hydroxymethyl)-methylamine salt or a polyamino alcohol salt of an unsaturated fatty acid, preferably an omega-3 or omega-6 fatty acid, or a mixture of salts as hereinbefore described for use in medicine or as a nutritional supplement.

Viewed from another aspect the invention provides the use of a water soluble tris-(hydroxymethyl)-methylamine salt or a polyamino alcohol salt of an unsaturated fatty acid, preferably an omega-3 or omega-6 fatty acid, or a mixture of salts as hereinbefore described in the manufacture of a medicament for use in medicine or as a nutritional supplement.

Thus, as well as certain new individual salts, the invention provides a mixture of water soluble amino alcohol salts of unsaturated fatty acids. By mixture here is meant that at least two different compounds are present and it will be appreciated that there are two possible variables, the nature of the salt component and the nature of the unsaturated fatty acid. A mixture as defined herein thus covers for example, a mixture of a meglumine salt of DHA and a meglumine salt of EPA. The mixture also covers different salts of the same acid, e.g. the mixture of a meglumine salt of DHA and a chitosan salt of DHA. The mixture also covers different salts of different acids, e.g. meglumine salt of DHA and a chitosan salt of EPA.

Preferably, the mixture comprises different unsaturated fatty acids but the same salt forming material.

The invention covers a mixture of water soluble amino alcohol salts of unsaturated fatty acids however made.

The crude composition which contains the fatty acids in acid form (COOH) or salt form preferably derives from the hydrolysis of a natural unsaturated fatty acid derivative containing oil (termed a crude oil herein). As noted above and as described in greater detail below, unsaturated fatty acids are generally present in triglyceride form in the natural environment so to release them into a free acid form requires hydrolysis of the ester bonds of the glyceride. It will be appreciated that the hydrolysed oil contains impurities and may therefore form the crude composition comprising said acid required of the invention.

The hydrolysis of the naturally occurring unsaturated fatty acid containing material can be carried out as described below and can involve a water wash of the hydrolysed material. This process forms a further aspect of the invention.

Thus, viewed from a further aspect the invention provides a process for the preparation of a water soluble unsaturated fatty acid salt containing composition comprising:
(I) hydrolysing a crude oil, e.g. a crude marine oil, containing at least one unsaturated fatty acid derivative and forming an oil phase containing at least one fatty acid;
(II) optionally washing the resulting oil phase with an aqueous wash;
(III) adding at least one amino alcohol compound to the product of step (I) or, if carried out, the product of step (II) in the presence of water so as to form a water soluble salt with said at least one unsaturated fatty acid thereby extracting said salt into the aqueous phase; and
(IV) isolating and optionally drying the aqueous phase.

In the first stage of the above process therefore, the crude, preferably marine, oil is hydrolysed to convert fatty acid derivatives therein (typically triglyceride compounds) into the carboxyl form COO⁻. Whilst this hydrolysis reaction could take place in acid, such conditions tend to isomerise double bonds present in the fatty acids and are not therefore preferred. Instead, the hydrolysis is preferably carried out in basic conditions. Such a reaction is a saponification reaction well known in chemistry.

Thus, in a typical saponification reaction as employable in the invention a hydroxide such as KOH, LiOH or NaOH reacts with trigylcerides present in the crude oil to give unsaturated fatty acid salts (typically Na, Li or K salts) and glycerol. Saponification can be carried out under conditions well known in the art, e.g. at elevated temperature. High pressures and steam may also be employed as is known.

Preferred saponification conditions involve the use of a polar solvent such as an alcohol with the aqueous base. After saponification in the presence of such a solvent mixture, the formed free fatty acid salts may partition into the aqueous phase as they exhibit solubility in alcohols such as ethanol. This allows non saponified material to be washed away using a non polar solvent wash, e.g. with hexane. The aqueous phase can then be acidified to form the free acids which are no longer soluble in the reaction medium.

The longer chain fatty acids released during the hydrolysis reaction thus form an oil phase and are thus separated from the glycerol and from the aqueous reaction medium. Shorter chain fatty acids, e.g. those of C10 or less, are water soluble in the aqueous phase, as for example, potassium or sodium salts and are thus separated from the non-water soluble longer chain components.

Since the saponification reaction may involve large quantities of aqueous material and a correspondingly low amount of oil phase material, the oil phase can be made easier to handle at this point e.g. by making it more voluminous, by adding a non-toxic organic solvent (often denoted a “a green chemistry solvent”) to dissolve the oil phase from the saponification reaction. The inclusion of this step has been found to improve the purification process. Suitable non toxic solvents are those which are capable of both dissolving the oil (i.e. essentially non-polar solvents) without being hazardous. Such solvents include alkanes such as pentane, hexane, ethers, acetates, ketones, xylene and toluene. Hexane and toluene are most preferred.

In a highly preferred embodiment therefore step (1) of the process described above involves:
(I) hydrolysing a crude oil containing at least one unsaturated fatty acid derivative in the presence of water and a polar organic solvent such an alcohol;
   washing the hydrolysed crude oil with a non polar solvent such as an alkane and retaining the aqueous phase;
   acidifying the aqueous phase to form an oil phase; and preferably adding a non polar solvent to said oil phase.

The oil phase and water phases can then, if desired, be separated by conventional techniques.

After the water phase is separated off, it is preferred if the oil phase is then washed with an aqueous solution as described in detail below. This is not however essential. As discussed in greater detail in the passages that follow, the washing step helps to remove certain water soluble components from the hydrolysed crude oil thus enriching the amount of unsaturated fatty acid present. However, since the hydrolysis reaction is conducted in aqueous solution, the separation of the oil phase from the aqueous phase during or after hydrolysis does itself effectively act as a washing step as water soluble components are retained in the aqueous phase. Nevertheless, in order to maximise the removal of water soluble components, further dedicated washing steps can be employed.

Further washing steps may be carried out using pure water or may be effected using a basic aqueous solution, e.g. dilute NaOH. During this washing stage (or during hydrolysis as the oil phase forms), shorter chain fatty acid compounds are removed (e.g. those of 10 carbon atoms or less) as these tend to be quite water soluble as potassium or sodium salts whereas longer chain fatty acids (e.g. those containing 18 carbon atoms or more) are not.

It will be appreciated however that solubility is dependent on chain length, ionic strength, pH, temperature etc and whilst very short chain fatty acids in the COO⁻ form will dissolve readily in water or in dil. NaOH solution and are therefore removed in an aqueous wash, medium chain fatty acids (e.g. C14 to C16) may be only partially soluble.

The aqueous washing also removes heavy metal contaminants from the hydrolysed crude oil as these tend to be water soluble. As heavy metal contamination is associated with diseases such as cancer, it is important to remove these compounds. Heavy metals could also be removed by addition of suitable complexing agents such as EDTA or DTPA.

Washing with water is effected conventionally with filtration, a separating funnel or continuous extraction being used to isolate the desired non-dissolved oil phase residues. The washing step can be carried out repeatedly if necessary.

The residue which is left after the washing stage primarily contains longer chain molecules (e.g. C18 or above), typically in salt form (e.g. sodium salt form). Since considerable amounts of shorter chain fatty acids are removed, and the majority of the highly desirable omega-3 fatty acids have at least 18 carbon atoms, the residue produced after at this stage is by definition enriched in desirable unsaturated fatty acid compounds such as omega-3 compounds.

It is the longer chain omega-3 molecules which provide the beneficial therapeutic effect and which ideally need to be present in a final omega-3 enriched composition. The oily residue after washing is not however, in a form suitable for administration as these longer chain fatty acid compounds are not water soluble. A medicament containing an omega-3 compound in its acid form or non soluble salt form is therefore of limited interest as large amounts of the omega-3 in the medicament will not dissolve in body fluids and will not be absorbed. If they are not absorbed, they cannot provide therapeutic benefit.

The residue formed after saponification and washing may also contain PCB's, flame retardants and dioxins as these are non water soluble.

The inventors have found that by forming the longer chain fatty acids into amino alcohol salts (in particular polyamino alcohol salts) the resulting salts are water soluble, and can therefore be formulated readily into useful medicaments or as nutraceuticals or pharmaceuticals. Moreover, water soluble salts can be extracted in an aqueous phase thereby being isolated from the impurities present in the crude oil which are not water soluble.

This technique also separates the fatty acid material from cholesterol which may be present in the crude composition or from any other lipophilic material e.g. phthalates. High cholesterol levels are well known to be associated with various diseases so removal of cholesterol from the fatty acids is also of great value. Like various of the other poisons mentioned above, cholesterol is water insoluble meaning it can be separated from the fatty acids during the salt formation step.

Since the oil phase formed up to this point (i.e. just prior to salt formation) may contain fatty acid in salt form, it is preferred if before conducting a further salt formation step, the longer chain fatty acid salts are returned to their acid form. This can be readily achieved in dilute acid. The fatty acids in acid form are actually more hydrophobic so will again readily form an oil phase at this point allowing any water soluble salts formed during the salt formation process to be simply removed in the aqueous phase.

Thus, the carboxylic acid groups of any unsaturated fatty acid salts in the oil phase can be converted to the COOH form using an acidic water phase. This is preferable to allow the fatty acids to form amino salts with the amino alcohol compound. This step can be followed by an organic wash with a non toxic solvent if desired. The composition formed at this stage is the preferred crude composition used in the process of the invention.

The salt formation reaction itself has been described above and requires only that the fatty acid or salt be brought into contact with the amino alcohol compound in the presence of water. Preferably the amino alcohol compound is added in aqueous solution although it will be appreciated that what is required here is that the amino alcohol compound is present with the fatty acid in the presence of an aqueous phase in which the water soluble salt which forms can be extracted.

As the amino alcohol salts form, they are extracted into the aqueous phase of the reaction medium leaving non water soluble impurities behind in the oil phase. The aqueous phase can then be isolated by standard phase separation techniques to provide a composition free of non water soluble impurities. In particular common poisons such as PCB's, flame retardants and dioxins are not water soluble so will not be extracted into the water phase. Cholesterol is also removed. This forms a further and important feature of the invention where the unsaturated fatty acid salt is for consumption.

Thus, viewed from another aspect the invention provides a composition comprising a water soluble amino alcohol salt of an unsaturated fatty acid, especially an omega-3 fatty acid, free of dioxins, flame retardants, cholesterol and/or PCB's.

The level of PCBs and dioxins in crude marine oil can be several hundred nanograms per gram, and even processed commercial marine oils have content of dioxins or PCB's in the range 0.16-30 nanograms per kilogram, (See e.g. Saldeen, P. et al., Obstetrical and Gynecological Survey 59 (2004) 722-730.) After the process of the invention, levels are preferably undetectable.

The whole process described above forms a still yet further aspect of the invention. Thus, viewed from another aspect the invention provides a process for the preparation of an unsaturated fatty acid salt containing composition comprising:
(I) hydrolysing a crude oil, e.g. a marine oil, containing at least one unsaturated fatty acid derivative and forming an oil phase containing a fatty acid;
(II) optionally dissolving the formed oil phase in a non-toxic solvent e.g. hexane or toluene;
(III) optionally washing the oil phase, e.g. repeatedly, with an aqueous wash;
(IV) optionally, adding an acidic aqueous phase to ensure any fatty acid compounds present are in the COOH form, and separating off the aqueous phase;
(V) adding at least one amino alcohol compound to the oil phase of the previous step in the presence of water so as to form a water soluble amino alcohol salt with said at least one unsaturated fatty acid or salt thereby extracting said salt into the aqueous phase; and
(VI) isolating and optionally drying the aqueous phase.

In a preferred embodiment, the unsaturated fatty acid is an omega-3 fatty acid. In a further preferred embodiment the at least one fatty acid comprising a mixture of omega-3 and omega-6 fatty acids.

In a further optional step, saturated fatty acids can be separated from the target unsaturated fatty acid materials. As exemplified in the present specification, the saturated fatty acids can be removed from the unsaturated fatty acids, especially those acids with cis configuration bonds, using urea complexation. In solvents like ethanol, urea tends to form a linear tunnel around straight chain fatty acid chains and complex them, thereby making them insoluble in ethanol, while the unsaturated fatty acids remain in the solution. The solid material, i.e. the saturated fatty acid/urea complex may then be removed by filtration.

The process of the invention may therefore include a step in which the oil phase is dissolved in alcoholic solvent, e.g. ethanol, and urea added. The solution can then be cooled if necessary and the crystalline product which forms separated. This is the undesirable saturated fatty acid component. The ethanol can then be removed from the remaining residue and the oil phase which remains used in the further process steps, perhaps after redissolution of the oil phase in a non toxic organic solvent to give it bulk.

This urea complexation step can conveniently occur just before salt formation, e.g. after neutralisation step (IV).

It will also be appreciated that the saturated fatty acids removed by urea complexation may themselves have useful end applications, e.g. in animal feeds, cosmetics etc. The saturated fatty acids which form the urea complex can be readily recovered using known procedures, e.g. by water extraction of the urea to leave a potentially valuable product. Whilst urea complexation is a well known reaction to isolate saturated fatty acids in this fashion, in conjunction with the saponification process described above forms a still yet further aspect of the invention.
(I) hydrolysing a crude oil, e.g. a marine oil, containing at least one unsaturated fatty acid derivative and at least one saturated fatty acid and forming an oil phase containing the fatty acids;
(II) optionally dissolving the formed oil phase in a non-toxic solvent e.g. hexane or toluene;
(III) optionally washing the oil phase, e.g. repeatedly, with an aqueous wash;
(IV) optionally, adding an acidic aqueous phase to ensure any fatty acid compounds present are in the COOH form, and separating off the aqueous phase;
(V) contacting the oil phase with urea in an alcoholic solvent and separating the crystalline product which forms;
(VI) recovering the saturated fatty acids in the crystalline product.

The unsaturated fatty acid isolation process described above is new and therefore allows the formation of salts and compositions which are also new, e.g. in view of their impurity profile. Viewed from another aspect, the invention provides a water soluble unsaturated fatty acid salt made by the process hereinbefore described.

Viewed from another aspect the invention provides a composition. e.g. a pharmaceutical composition, comprising a water soluble salt made by the process hereinbefore described.

Viewed from another aspect the invention provides a water soluble salt made by the process hereinbefore described for use in medicine or as a nutritional supplement.

The inventors have found that by forming the fatty acids into amino alcohol and/or polyamino alcohol salts (in particular polyamino alcohol salts) the resulting salts are water soluble, and can therefore be formulated readily into useful medicaments or as nutraceuticals or pharmaceuticals. Moreover, they can be extracted in an aqueous phase thereby being isolated from the poisons present in the marine oil which are not water soluble.

As the amino alcohol salts of the omega-3 fatty acids are water soluble they are more bioavailable meaning a medicament comprising such a compound can contain less active agent than those currently on the market where the omega-3 compounds are in a less bioavailable form. It is known from literature, e.g. Beckerman et al., Arzneimittelforschung 40 (1990) 700-704, that the bioavailability of free fatty acids, i.e. the form they will be in the acidic conditions in the stomach, lead to maximal 50% higher plasma levels than the origin marine oil in the form of triglycerides. This is yet another advantage of the resulting powder in the present invention compared to the state of art marine oils. Consequently, the dose of omega-3 can be significantly lower with the present invention because of higher bioavailability.

A further problem with unsaturated fatty acid materials is the potential for oxidation of the double bonds therein. If they are stored or formulated freely exposed to air or at higher temperatures, an unpleasant taste and odour can occur since oxidation can take place in air owing to the large number of carbon-carbon double bonds in their molecules. The result may be deterioration in time of the organoleptic characteristics and potential formation of epoxy groups and short chain aldehydes or carboxylic acids as the result.

To avoid this oxidative process, it is preferred if the unsaturated water soluble fatty acid salts are mixed with cyclodextrins, e.g. α-, β- and γ-cyclodextrin or hydroxypropyl-β-cyclodextrin, especially β-cyclodextrin.

Thus, the final aqueous solution of the amino alcohol salt of the fatty acid may be mixed with a cyclodextrin, e.g. α-, β- and γ-cyclodextrin or hydroxypropyl-β-cyclodextrin, to form a heterogeneous mixture. This may be stirred for a period of 1 to 24 hours at a temperature of between 0° and 100° C. to form a complex between the salt and cyclodextrin. This can then be isolated by the techniques described below, e.g. vacuum evaporation of solvent, lyophilisation or spray drying. The products obtained are water-soluble, yellowish or white, crispy and have virtually no fishy taste or smell.

The amount of cyclodextrin added can vary but a suitable ratio of fatty acid salt to cyclodextrin is 2:1 to 1:2 by weight.

Viewed from a further aspect therefore the invention provides a composition comprising a water soluble amino alcohol salt of an unsaturated fatty acid and at least one cyclodextrin. The composition may be a complex of these materials.

One or more physiologically tolerable antioxidants may be additionally or alternatively added to the aqueous phase containing the desired amino alcohol salts in order to prevent their degradation. These may be added either before or, more preferably, after isolation of the aqueous phase. Antioxidants suitable for use in the invention include both water-soluble and oil soluble compounds and combinations thereof, however water-soluble antioxidants such as ascorbic acid (especially L-ascorbic acid) and citric acid are generally preferred. Oil soluble antioxidants which may be employed include α-tocopherol (vitamin E). Desired amounts of antioxidant may be readily determined by those skilled in the art but may be of the order of 0.5 to 10 wt % relative to the fatty acid salt.

Compositions comprising at least one antioxidant form a further aspect of the invention. Thus, viewed from a further aspect the invention provides a composition comprising a water soluble amino alcohol salt (preferably a polyamino alcohol salt) of an unsaturated fatty acid and at least one physiologically tolerable antioxidant. More preferably, the invention provides such compositions which are also substantially free of dioxins, flame retardants or PCB's.

A further advantage of amino alcohol salts, especially polyamino alcohol salts, is that they are believed to prevent isomerism of cis double bonds in the omega-3 or omega-6 material. Since the material can be isolated as a powder cis trans isomerism is much less of an issue than in a liquid oil. Exposure to light can cause double bond isomerization in oils but is much less likely to do so in a powder.

A most significant benefit therefore of the invention is that the fatty acid salts can be isolated in powder form, most preferably crystalline form, making their handling and formulation simple compared to an oil. Powders for example can be readily made into tablets which are preferable to the capsules needed to encase an oil and typically employed in the market place today.

To obtain the salt in powder form the aqueous phase need simply be evaporated however it is preferred if lyophilisation or spray drying is employed. The formation of the powder form of the salt is especially important where a mixture of salts is present as typically salt mixtures cannot be isolated as powders.

Thus, viewed from a further aspect the invention provides a process for the preparation of a powder, preferably crystalline, mixture of water soluble amino alcohol salts of unsaturated fatty acids (preferably the same salt of different fatty acids) comprising spray drying or lyophilising an aqueous solution of a mixture of water soluble amino alcohol salts of unsaturated fatty acids.

Spray drying techniques are disclosed in “Spray Drying Handbook”, K. Masters, 5th edition, Longman Scientific Technical UK, 1991, the disclosure of which is hereby incorporated by reference at least for its teaching of spray drying methods. Examples of the use of spray drying to produce powder of fatty acid based products can be found in prior art, e.g. in U.S. Pat. No. 5,106,639. Water solutions of the salts as described in the present invention may heated to a temperature of from 20 to 50° C. and dried in a spray drier using blown air of from 50 to 180° C.

Lyophilisation may be carried out by conventional methods. However, it may be advantageous to include one or more agents having a cryoprotective effect when performing this procedure. Any physiologically tolerable cryoprotectant may be used and examples of such agents are well known in the art. These include, for example, polyols such as glycerol; aminoacids such as glycine; carbohydrates, e.g. sugars such as sucrose, mannitol, trehalose, glucose, lactose or a polysaccharide such as dextran. Physiologically well-tolerated sugars, such as sucrose, are particularly preferred. Desired amounts of cryoprotectant may be readily determined by those skilled in the art but may be of the order of 0.5 to 10 wt % relative to the fatty acid salt.

Lyophilised products formed in the presence of one or more cryoprotective agents form a further aspect of the invention. Viewed from another aspect the invention thus provides a composition (preferably a lyophilised composition) comprising a water soluble amino alcohol salt (e.g. a polyamino alcohol salt) of an unsaturated fatty acid and at least one physiologically tolerable cryoprotective agent. More preferably, the invention provides such compositions which further include at least one physiologically tolerable antioxidant, cyclodextrin and/or which are also substantially free of dioxins, flame retardants or PCB's.

In a further embodiment of the invention the drying step can be carried out by lyophilisation or spray drying.

In a highly preferred embodiment therefore the process of the invention comprises at least the steps:
(A) hydrolysing a crude oil, e.g. a marine oil, containing at least one unsaturated, preferably omega-3 fatty acid derivative;
(B) adding an acidic aqueous phase to convert said at least one omega-3 fatty acid into its COOH form, and separating off the aqueous phase;
(C) adding an aqueous phase comprising at least one amino alcohol compound so as to form a water soluble salt with said at least one unsaturated, preferably omega-3, fatty acid thereby extracting said salt into the aqueous phase.

The salts of the unsaturated fatty acids produced by the process of the invention, especially omega-3 and omega-6 salts can be used directly in health supplements. They can be formulated conventionally into medicaments using conventional techniques well known to the skilled pharmaceutical chemist. Thus, the compounds may be formulated with well known recipients or conventional additives such as antioxidants, preservatives, colouring, flavouring etc.

The compounds of the invention may be formulated in any convenient form such as tablets, coated tablets, pills, powder, capsules, emulsions, creams, pessiaries, suppositories etc. The mode of administration may be any known mode, such as oral, nasal, transmucosal, parenteral, topical, intradermal etc. Oral administration is preferred. Since the process of the invention provides high purity omega-3 compounds in high yield, it is possible to provide the omega-3 supplement in a “once a day” composition. Such a composition should comprise 100 to 300 mg of omega-3 material.

In a preferred embodiment, the enriched omega-3 composition is formulated with phospholipids and/or monosaccharides to add water solubility and tablet quality.

The omega-3 salts produced using the process of the invention can be employed in the treatment and/or prevention of any condition in which omega-3 has been implicated to help. Such conditions include autoimmune disorders, inflammation, stroke, hypertension, skin disorders, cancer, brain and retina function, neurological disorders, infant growth and development and in particular in hearth health. Omega 3 may lower triacylgylycerol levels, may lower low density lipoprotein cholesterol levels, lowering incidence of arrhythmia, lower atherosclerosis/thrombosis. Thus, the use of omega 3 of this invention in the manufacture of a medicament for the treatment or prevention of any of these conditions forms a further aspect of the invention.

The invention has been described in relation to unsaturated fatty acid compounds as these are known to provide a useful therapeutic benefit. It will be appreciated however that the technique described herein can also be used to isolate saturated fatty acid compounds using exactly the same principles. These materials are used in animal feeds so the ability to obtain these materials in water soluble form and powder form is also invaluable.

Viewed from another aspect therefore the invention provides a process for the preparation of a water soluble saturated fatty acid salt from a crude composition comprising at least one non-water soluble or sparingly water soluble saturated fatty acid or salt thereof, said process comprising:

adding to said crude composition in the presence of water at least one amino alcohol compound so as to form a water soluble amino alcohol salt of said acid or salt;

separating the aqueous phase; and

optionally isolating said salt from said aqueous phase.

Viewed from another aspect the invention provides a process for the preparation of a water soluble saturated fatty acid salt containing composition comprising:
(I) hydrolysing a crude oil, e.g. a crude marine oil, containing at least one saturated fatty acid derivative and forming an oil phase containing the fatty acid;
(II) optionally washing the resulting oil phase with an aqueous wash;
(III) adding at least one amino alcohol compound to the product of step (1) or, if carried out, the product of step (II) in the presence of water so as to form a water soluble salt with said at least one saturated fatty acid thereby extracting said salt into the aqueous phase; and
(IV) isolating and optionally drying the aqueous phase.

Viewed from another aspect, the invention provides a water soluble amino alcohol salt of a saturated fatty acid, preferably one comprising at least 10 carbon atoms, especially at least 12 carbon atoms.

Viewed from another aspect, the invention provides a composition, e.g. a pharmaceutical composition, comprising an amino alcohol salt of a saturated fatty acid.

Viewed from another aspect the invention provides amino alcohol salt of an saturated fatty acid for use in medicine or as a nutritional supplement.

The preferred salts and process features described above apply to the isolation of saturated fatty acids as well although a urea complexation step is obviously not desirable. Preferred saturated fatty acid salts which can be formed in this fashion include those of C10 or more, e.g. C12 or more, preferably C14 or more, especially C16 or more. The carbon backbone is preferably linear.

The invention will now be described with reference to the following non-limiting examples.

### EXAMPLE 1

Preparation of DHA Salt with Chitosan (5:1)

A solution of chitosan acetate (100 mg; 17 μmol) in de-ionized water (4 mL) was passed through a column of Amberlyst® A-26(OH) resin (1 g). The resin was rinsed with de-ionized water (10 mL), A solution of (all cis)-4,7,10,13,16,19-docosahexaenoic acid (28 mg; 85 μmol) in 96% ethanol (0.5 mL) was added to the combined eluates, giving a cloudy solution. After freeze-drying, 53 mg off-white solid was obtained, which charred without melting when heated to 350° C. The product was stored in a freezer.

### EXAMPLE 2

Preparation of DHA Salt with Chitosan (20:1)

The previous procedure was repeated using chitosan acetate (100 mg; 17 μmol) and ((all cis)-4,7,10,13,16,19-docosahexaenoic acid (112 mg; 340 μmol). Freeze-drying gave 131 mg of solid.

### EXAMPLE 3

Tablet Preparation

Tablet comprising DHA salt (Example 1) was prepared by direct compression of a mixture of DHA salt (25 mg), microcrystalline cellulose (Avicel) (140 mg) and lactose monohydrate (135 mg). Tablet weight 300 mg. Tablet diameter 7 mm.

### EXAMPLE 4

### Purification of DHA (cis-4,7,10,13,16,19-Docosahexaenoic Acid)

A solution of (all-cis)-4,7,10,13,16,19-docosahexaenoic acid (DHA) (100 mg; 0.30 mmol) and 2,4,5-trichlorobiphenyl (1.0 mg; 3.9 mmol) in toluene (10 mL) was extracted with a solution of N-methylglucamine (0.5 g; 2.6 mmol) in water (5 mL). The yellow emulsion was centrifuged for ca. 12 min. GLC analysis of the toluene phase indicated that only the trichlorobiphenyl was present. The aqueous phase was acidified with 1 M HCl (5 mL) and extracted with toluene (1×0.5 mL). GLC analysis of the extract indicated that it contained 0.25% PCB, 98.5% DHA, and 1.3% other impurities. After repeating the salt formation process and reisolating the free fatty acid, the level of PCB compounds become too low to be detected.

### EXAMPLE 5

### Purification of Free Fatty Acids from Seal Blubber Oil by Extraction with Meglumine

KOH (5.75 g) was added to a mixture of seal blubber oil (SBO) (25 g) and decabromodiphenyl ether (1.25 g) in H₂O (11 ml) and MeOH (66 ml). The mixture was heated to reflux overnight under argon atmosphere, cooled to room temperature and the saponified mixture diluted with H₂O (50 ml). The non-saponified matter was extracted into n-hexane (2×100 ml) and discarded. The aqueous layer was acidified with 2 M HCl to pH 2 and the free fatty acids (FFAs) extracted into n-hexane (2×50 ml). The organic layer was dried over anhydrous Na₂SO₄ and the solvent removed at 40° C. in vacuo to leave the FFAs as yellow oils.

Urea (20 g) was added to a sample of FFAs (10 g) dissolved in 96% aqueous EtOH (100 ml). The mixture was stirred and heated until clear. The solution was kept in the freezer overnight. The urea complex was filtered from the liquid. The filtrate was diluted with an equal volume of water and acidified with 2 M HCl to pH 2. n-Hexane (100 ml) was added to the aqueous mixture and stirred for ½ h. The organic layer was separated and extracted with a 10% aqueous meglumine solution (2×50 ml). The organic layer was separated and discarded. The aqueous meglumine solution washed with n-hexane (2×50 ml), then acidified with 2 M HCl to pH 2. The FFAs were extracted into n-hexane (2×50 ml), dried over anhydrous Na₂SO₄ and the solvent evaporated in vacuo. GC analysis of the FFAs indicated that it did not contain any decabromodiphenyl ether.

### EXAMPLE 6

Preparation of EPA Salt with Chitosan and Added Water Soluble Antioxidants

Chitosan powder (low molecular weight) (2 g) was added to a stirred solution of cis-5,8,11,14,17-eicosapentaenoic acid (EPA) (0.5 g) in DMSO (5 ml) and H₂O (10 ml) under argon atmosphere at room temperature. L-Ascorbic acid (100 mg) and citric acid (100 mg) were added and the mixture stirred for 2 h, then freeze-dried overnight to leave the product as a yellow solid.

### EXAMPLE 7

Preparation of Free Fatty Acid (FFA) Salt with Chitosan and Added Water Soluble Antioxidants

Chitosan powder (low molecular weight) (2 g) was added to a stirred solution of FFAs (obtained by meglumine extraction of FFAs from Seal Blubber Oil) (0.5 g) in DMSO (5 ml) and H₂O (10 ml) under argon atmosphere at room temperature. L-Ascorbic acid (100 mg) and citric acid (100 mg) were added and the mixture stirred for 2 h, then freeze-dried overnight to leave the product as a yellow solid.

### EXAMPLE 8

Preparation of EPA Chitosan Salt with Added Water Soluble Antioxidants and α-Tocopherol

Chitosan powder (low molecular weight) (2 g) was added to a stirred solution of EPA (0.5 g) in DMSO (5 ml) and 1H₂O (10 ml) under argon atmosphere at room temperature. L-Ascorbic acid (100 mg), citric acid (100 mg) and α-tocopherol (100 mg) were added and the mixture stirred for 2 h, then freeze-dried overnight to leave the product as a yellow solid.

### EXAMPLE 9

Thermally stabilised EPA meglumine salt with added water soluble antioxidants and α-Tocopherol

A solution of EPA (1.0 g) in n-hexane (10 ml) was extracted with a 10% aqueous meglumine solution (30 ml). The aqueous meglumine solution was transferred to an erlenmeyer flask and L-ascorbic acid, citric acid and α-tocopherol (50 mg of each) were added and stirred vigorously. The aqueous mixture was split into 3×10 ml samples and transferred to 500 ml round bottom flasks. The samples were treated as follows: a) no cryoprotectant added; b) 3.3 g sucrose added; and c) 6.6 g sucrose added prior to freeze-drying. The resulting mixtures were freeze-dried overnight to leave the products as crystalline solids.

### EXAMPLE 10

Thermally Stabilised Free Fatty Acid (FFA) Meglumine Salt with Added Water Soluble Antioxidants and α-Tocopherol

Following the procedure outlined in Example 5, crude seal oil (from GC Rieber Oils AS) was hydrolyzed to yield FFAs. A sample of the FFAs (1.0 g) in n-hexane (10 ml) was extracted with a 10% aqueous meglumine solution (30 ml). The aqueous meglumine solution was added to L-ascorbic acid, citric acid and α-tocopherol (50 mg of each) and stirred vigorously. The aqueous mixture was split into 3×10 ml samples and transferred to 500 ml round bottom flasks. The samples were treated as follows: a) 3.3 g sucrose added; b) 6.6 g sucrose added; and c) 9.9 g sucrose added prior to freeze-drying. The resulting mixtures were freeze-dried overnight to leave the products as crystalline solids.

### EXAMPLE 11

Extraction of Different Fatty Acids with Meglumine, Tris(Hydroxymethyl)Aminomethane and NaOH

A mixture of decanoic acid (1.0 g), lauric acid (1.0 g), myristic acid (1.0 g), palmitic acid (1.0 g), stearic acid (1.0 g) and arachidic acid (1.0 g) in n-Hexane (200 ml) washed with a 1 M aqueous NaOH solution (3×50 ml). The aqueous NaOH solution was acidified with 2 M HCl to pH 2, and the FFAs extracted into n-Hexane (2×100 ml). GLC analysis of the n-Hexane extracts indicated that NaOH could be used for extraction of decanoic acid (C₁₀H₂₀O₂), but not for FFAs with longer chain lengths.

The experiment was repeated, but the organic layer washed with a 10% aqueous meglumine solution and a 10% tris(hydroxymethyl)aminomethane solution respectively. Extraction with meglumine and (tris(hydroxymethyl)aminomethane) showed that all FFAs was extracted from the organic layer into the aqueous solution.

### EXAMPLE 12

Thermally Stabilised FFA Meglumine Salt

N-Methylglucamine (0.57 g) was added to a stirred emulsion of FFAs (obtained from example 5) (0.5 g) in H₂O (30 ml) The resulting aqueous meglumine solution was transferred to a 500 ml round bottom flask and freeze-dried overnight to leave the products as a semi-crystalline solid.

### EXAMPLE 13

Thermally Stabilised FFA Meglumine Salt Added β-Cyclodextrin

N-Methylglucamine (3.0 g) and β-cyclodextrin (1.0 g) was added to a stirred emulsion of FFAs (obtained from example 5) (0.5 g) in H₂O (30 ml) The resulting aqueous meglumine solution was transferred to a 500 ml round bottom flask and freeze-dried overnight to leave the products as a white powder.

### EXAMPLE 14

Thermally Stabilised FFA Meglumine Salt Added β-Cyclodextrin and L-Ascorbic Acid

N-Methylglucamine (0.57 g) and β-cyclodextrin (0.5 g) was added to a stirred emulsion of FFAs (obtained from example 5) (0.5 g) in H₂O (30 ml) The resulting aqueous meglumine solution was added L-ascorbic acid (50 mg), transferred to a 500 ml round bottom flask and freeze-dried overnight to leave the products as a yellow powder.

### EXAMPLE 15

Thermally Stabilised FFA Tris Salt Added β-Cyclodextrin

Tris(hydroxymethyl)aminomethane (0.3 g) and β-cyclodextrin (0.5 g) was added to a stirred emulsion of FFAs (obtained from example 5) (0.5 g) in H₂O (30 ml) The resulting aqueous tris solution was transferred to a 500 ml round bottom flask and freeze-dried overnight to leave the products as a white powder.

### EXAMPLE 16

Thermally Stabilised FFA Meglumine Salt Prepared Under Non-Aqueous Conditions

A mixture of FFAs (obtained from example 5) (0.5 g) and n-methylglucamine (0.37 g) in MeOH (40 ml) was refluxed at 60° C. for 2 h, cooled to room temperature and evaporated in vacuo to leave the FFA meglumine salt as a semi-crystalline solid.

### EXAMPLE 17

Solubility of FFA Meglumine Salt and FFA Tris Salt Added β-Cyclodextrine

The solubility of the FFA meglumine salt (from example 12) and the FFA tris salt (from example 15) was determined by adding the salt to vials of H₂O (1.0 ml). The solubility of the FFA megluine salt was determined to be approximately 650 mg/ml. The solubility of the FFA tris salt was determined to be approximately 50 mg/ml.

### EXAMPLE 18

Preparation of FFA Esters and Other Derivatives

The FFA mixture from Example 5 is re-isolated by acidification of a water solution comprising the amino alcohol salt, extraction into a suitable organic green solvents e.g. hexane or toluene and evaporation of the solvent. The isolated FFA mixture is dissolved in dry ethanol. Catalytic amounts of hydrochloric acid are added and the solution is stirred at ambient temperature for 24 hours. During this period, a part of the solvent is distilled off, and new solvent is added to the original volume. The solution is evaporated, and the mixture of fatty acid esters is isolated by molecular distillation.

It should be apparent that the foregoing relates only to the preferred embodiments of the present application and that numerous changes and modification may be made by one of ordinary skill in the art without departing from the general spirit and scope of the invention as defined by the following claims and the equivalents thereof.

1. A process for the preparation of a water soluble unsaturated fatty acid salt from a crude composition comprising at least one non-water soluble or sparingly water soluble unsaturated fatty acid or salt thereof said process comprising:
adding to said crude composition in the presence of water at least one amino alcohol compound so as to form a water soluble amino alcohol salt of said acid or salt;
separating the aqueous phase; and
optionally isolating said salt from said aqueous phase.

2. A process as claimed in claim 1 for the preparation of a mixture of water soluble unsaturated fatty acid salts from a non-water soluble or sparingly water soluble mixture of unsaturated fatty acids or salts thereof, said process comprising:
adding to said mixture of non-water soluble or sparingly water soluble unsaturated fatty acids or salts thereof in the presence of water at least one amino alcohol compound so as to form a water soluble amino alcohol salt of said acids or salts;
separating the aqueous phase; and
optionally isolating said salt from said aqueous phase.

3. A process as claimed in claim 1 for the preparation of a water soluble unsaturated fatty acid salt from a non-water soluble or sparingly water soluble unsaturated fatty acid or salt thereof, said process comprising:
adding to said non-water soluble or sparingly water soluble unsaturated fatty acid or salt thereof in the presence of water at least one polyamino alcohol compound so as to form a water soluble polyamino alcohol salt of said acid or salt;
separating the aqueous phase; and
optionally isolating said salt from said aqueous phase.

4. A process as claimed in claim 1 wherein said crude composition derives from a crude marine oil.

5. A process for the preparation of a water soluble unsaturated fatty acid salt as claimed in claim 1 comprising:
(I) hydrolysing a crude oil containing at least one unsaturated fatty acid derivative and forming an oil phase comprising said fatty acid;
(II) optionally washing the resulting oil phase with an aqueous wash;
(III) adding at least one amino alcohol compound to the product of step (I) or, if carried out, the product of step (II) in the presence of water so as to form a water soluble salt with said at least one unsaturated fatty acid thereby extracting said salt into the aqueous phase; and
(IV) isolating and optionally drying the aqueous phase.

6. A process for the preparation of an unsaturated fatty acid salt as claimed in claim 5 comprising:
(I) hydrolysing a crude oil containing at least one unsaturated fatty acid derivative and forming an oil phase comprising said fatty acid;
(II) dissolving the formed oil phase in a non-toxic solvent e.g. hexane or toluene;
(III) optionally washing the oil phase, e.g. repeatedly, with an aqueous wash;
(IV) optionally, adding an acidic aqueous phase to ensure any fatty acid compounds present are in the COOH form, and separating off the aqueous phase;
(V) adding at least one amino alcohol compound to the oil phase of the previous step in the presence of water so as to form a water soluble amino alcohol salt with said at least one unsaturated fatty acid or salt thereby extracting said salt into the aqueous phase; and
(VI) isolating and optionally drying the aqueous phase.

7. A process as claimed in claim 5 wherein the hydrolysis reaction is carried out under basic conditions.

8. A process as claimed in claim 7 wherein the hydrolysis reaction takes place in the presence of an alcoholic solvent.

9. A process as claimed in claim 5 wherein step (I) involves:
(I) hydrolysing a crude oil containing at least one unsaturated fatty acid derivative in the presence of water and a polar organic solvent such an alcohol;
washing the hydrolysed crude oil with a non polar solvent such as an alkane and retaining the aqueous phase;
acidifying the aqueous phase to form an oil phase.

10. A process as claimed in claim 1 wherein at least one of polychlorinated biphenyls, dioxins, heavy metals, flame retardants, phthalates and cholesterol are present in the crude composition and are removed during the salt formation process.

11. A process as claimed in claim 1 wherein a polyamino alcohol compound is used.

12. A process as claimed in claim 11 wherein said polyamino alcohol compound is chitosan.

13. A process as claimed in claim 1, wherein a monoamino alcohol compound is used.

14. A process as claimed in claim 13 wherein said monoamine compound is meglumine or tris(hydroxymethyl)aminomethane.

15. A process as claimed in claim 1 wherein a mixture of unsaturated fatty acids is present in the crude composition.

16. A process as claimed in claim 1 wherein the water soluble unsaturated fatty acid salt is an omega-3 unsaturated fatty acid salt, is a mixture of a omega-3 unsaturated fatty acid salts or is a mixture of an omega-3 unsaturated fatty acid salt and an omega-6 unsaturated fatty acid salt.

17. A process as claimed in claim 1 to 16 wherein the unsaturated fatty acid is or is derived from a compound of formula (I)
CH₃(CH₂)ₙ—(CH═CH—CH₂)ₘ—(CH₂)ₛ—COOH (I)
wherein n, m and s are integers.

18. A process as claimed in claim 1 wherein the salt is isolated by drying and after drying, a solid is formed.

19. A process as claimed in claim 1 wherein drying is effected by spray drying or lyophilisation.

20. A process as claimed in claim 1 for the preparation of an omega-3 unsaturated fatty acid salt containing composition comprising:
(A) hydrolysing a crude oil, e.g. a marine oil, containing at least one omega-3 fatty acid or derivative thereof;
(B) adding an acidic aqueous phase to convert said at least one omega-3 fatty acid into its COOH form, and separating off the aqueous phase;
(C) adding an aqueous phase comprising at least one monoamino and/or polyamino alcohol compound so as to form a salt with said at least one omega-3 fatty acid thereby extracting said salt into the aqueous phase.

21. A water soluble amino alcohol salt of an unsaturated fatty acid or composition comprising a mixture of such salts made by a process as claimed in claim 1.

22. A water soluble tris-(hydroxymethyl)-methylamine salt or a polyamino alcohol salt of an unsaturated fatty acid.

23. A composition comprising a mixture of water soluble amino alcohol salts of unsaturated fatty acids.

24. (canceled)

25. A composition as claimed in claim 23 additionally comprising at least one physiologically tolerable antioxidant.

26. A composition as claimed in claim 23 additionally comprising at least one physiologically tolerable cryoprotective agent.

27. A composition as claimed in claim 23 additionally comprising at least one cyclodextrin.

28. A composition as claimed in claim 23 in powder form.

29. A composition as claimed in claim 23 in crystalline form.

30. A composition as claimed in claim 23 wherein at least one fatty acid salt therein is derived from an acid of formula (I′)
CH₃CH₂CH═CH—R—COOH (I′)
wherein R is a C₁₃₊ alkylene group (e.g. C₁₃₋₂₅) optionally containing 1 or more double bonds, preferably non-conjugated; or
formula (II):
CH₃CH₂CH₂CH₂CH₂CH═CH—R″—COOH (II)
wherein R″ is a C₅₊ alkylene group (e.g. C₁₀₋₂₂) optionally containing 1 or more double bonds.

31. A composition as claimed in claim 30 wherein a fatty acid salt derived from a compound of formula (I′) and (II) is present.

32. A composition as claimed in claim 31 comprising an amino alcohol salt of DHA, EPA, linoleic acid or conjugated linoleic acid or a mixture thereof.

33. A composition as claimed in claim 23 wherein the amino alcohol compound which forms the salt is of general formula
((HO(R′)_{0/1})ₙ)R₁NHR₂)ₘ (III)
where n is an integer from 3 to 6, R′ is methyl, R₁ is a C1-20 alkylene group, R₂ is H or an C₁₋₆alkyl side chain, preferably methyl and m is an integer from 1 to 20; or
general formula
(C₆H₁₄NO₅)ₚ (IV)
where p is an integer of 2 or more.

34. A composition as claimed in claim 33 wherein the amino alcohol compound is meglumine, tris(hydroxymethyl)aminomethane or chitosan.

35. A composition as claimed in claim 23 having a water solubility of at least 250 g/L.

36. A composition as claimed in claim 23 being a medicine or a nutritional supplement.

37. (canceled)

38. (canceled)

39. A process as claimed in claim 1 for the preparation of a water soluble saturated fatty acid salt containing composition comprising:
(I) hydrolysing a crude oil containing at least one saturated fatty acid derivative and forming an oil phase comprising said fatty acid;
(II) optionally washing the resulting oil phase with an aqueous wash;
(III) adding at least one amino alcohol compound to the product of step (I) or, if carried out, the product of step (II) in the presence of water so as to form a water soluble salt with said at least one saturated fatty acid thereby extracting said salt into the aqueous phase; and
(IV) isolating and optionally drying the aqueous phase.

40. A water soluble amino alcohol salt of a saturated fatty acid.

41. A composition comprising a mixture of water soluble amino alcohol salts of a saturated fatty acids.
